(19) United States
(12) Patent Application Publication
(10) Pub. No.: US 2025/0345368 A1
(43) Pub. Date: Nov. 13, 2025

(54) Herring Sperm DNA, as a means of causing Cancer Cells to go into a dormant state, or to stall or inhibit the growth or metastasis of the Cancer Cells

(71) Applicant: Paul Dwork, Encinitas, CA (US)

(72) Inventor: Paul Dwork, Encinitas, CA (US)

(21) Appl. No.: 19/275,766

(22) Filed: Jul. 21, 2025

Related U.S. Application Data
(60) Provisional application No. 63/675,400, filed on Jul. 25, 2024.

(51) Int. Cl. A61K 35/60; A61K 31/713

(52) U.S. Cl. CPC A61K 35/60; A61K 31/713

(57) ABSTRACT

Herring Sperm has been used as a blocking agent and in use as an anti-cancer. My discovery/invention is not an anti-cancer agent, but a way to stunt or minimize growth of the cancer cells, and potentially going into a temporary or dormant state, to allow current chemotherapy to be affective in cancer cell destruction, allowing time to work and supersede the cancer cell growth process, as well as stunting or slowing down potential metastasis.

### BACKGROUND AND SUMMARY

This discovery was made while working with the Herring Sperm, and evaluating its effect on the growth of cellular tissue, in a living organism, a fresh water fish called Poecilla reticulata. These are live bearing fish. It was discovered by studying, and testing that the cellular growth was stunted, which indicated the DNA had an unusual effect on the developing cellular tissue or cells.

The chemical name for the Herring Sperm, or the primary component of the Herring Sperm is deoxyribonucleic acid, (DNA), and is the molecule that carries the genetic information.

In particular Herring Sperm has a relatively high DNA content, when compared to sperm DNA of other fish, such as Salmon as an example. This unique property makes it unique and my discovery of this has never been considered.

Currently it is used in a variety of research, including DNA binding studies, physiochemical studies of DNA, and as a blocking agent in molecular biology techniques.

No current research, or review of published studies, have considered this unique approach.

### BRIEF SUMMARY

Cancer is a group of diseases that occur when cells divide uncontrollably and spread into surrounding tissues. These abnormal cells can also spread to other parts of the body (metastasis) through the blood and lymph systems and surrounding tissues. These cells spread uncontrollably, and if not stopped, restrained, and/or destroyed, the results are unpredictable, and potentially can lead to death. Cells go through different phases during their lives. These phases may involve preparing for cell division, or they may be in a phase where they are not moving toward cell division. This normal cycle when interrupted is abnormal and can lead to abnormal cells, as well as abnormal duplication, which in turn leads to cancer cells.

This patent, Discovery, will allow for the cells to potentially go dormant, decrease their duplication and possibly cause them to self-destruct, and allow normal cells to replicate and overwhelm them Chemotherapy medications when delivered, may have a chance to be more effective, during this dormant or latency phase, thereby having a greater efficacy in destroying the cancer cells

### DETAILED DESCRIPTION

By its nature, cancer has aberrant DNA that prompts proliferation of the cancerous tissue. Such proliferation exhibits unique stages and characteristics. Initial growth is manifested by angiogenesis which is the rapid growth of blood vessels-required to nourish the cancer. Also, cancer tissue with its high (blood) perfusion is characterized by leaky membranes, openings that allow for the egress of small particles that normally wouldn't enter non-cancerous tissues. These large openings, up to −2 micron, would allow exposure to the delivered DNA/sperm wherein they are retained within the cancer tissue. This enhanced permeability and retention (EPR) effect is well recognized within the scientific community. The morphology of intact sperm may lead itself may lead itself to be incorporated with the porous cancer tissue by way of the sperm head being only −2 micron in diameter and/or by entanglement into the openings of the sperm tail which is less than 0.5 micron in diameter and up to 50 microns in length. Should the tail enter these membrane openings, it may progressively integrate itself within the cancer by way of reptation motion. (referring to snake-like, creeping motion of long polymer chains through a network of other entangled polymer chains) with this formulation, and proper delivery into the tumor or systemic system causing the desired effect of causing the Cancer Cell to go into a dormant, regressive, inhibitory, interrupted cell replication, or shrinkage. The Herring Sperm or the isolated Active DNA component, when introduced to the cancer cells could temporarily or permanently pause the cancer cells from normal replication. Reduction in size, or stunting their growth, thereby allowing the chemotherapy protocols to take advantage and enhance their therapeutic benefit during this phase.

### SUMMARY CONCLUSION

This Utility patent application is specifically targeted to limit, stall, or inhibit the cancer cell growth, with Herring Sperm DNA. It is frequently used in scientific research, particularly for studies involving DNA binding, nucleic acid hybridization, and as a blocking agent in various molecular biology techniques. There have been no documented studies or investigations using Herring Sperm DNA for this purpose, and this is a new and unique use for the enhancement of Cancer Therapy outcomes. This will allow proper cancer drug protocols time to destroy cancer cells during this period of inhibition, potential dormancy, or stalling (referring to significantly slowing down) the cancer.

1. DNA extracted from Herring Sperm causing the replication process to be stunted in growth of Cancer Cells or irregular cells.

2. DNA extracted from Herring Sperm causing the causing inhibition of the normal process of cancer cell replication.

3. DNA extracted from Herring Sperm causing the replication process to be interrupted for the normal growth and replication process.

4. DNA extracted from Herring Sperm causing regression or shrinkage of the affected cellular network.

5. DNA extracted for Herring Sperm causing an interference of the chromosomal aberration and suppressing the growth or exacerbation of the altered cancer genomes, that control the cell growth, division, and potential death.
